(19) United States
(12) Patent Application Publication
(10) Pub. No.: US 2026/0335633 A1
(43) Pub. Date: Oct. 29, 2026

(54) REGENERATIVE PERIPHERAL NERVE INTERFACE DEVICE AND METHOD FOR MANUFACTURING SAME

(71) Applicants: DAEGU GYEONGBUK INSTITUTE OF SCIENCE AND TECHNOLOGY, Daegu (KR); THE ASAN FOUNDATION, Songpa-gu Seoul (KR); UNIVERSITY OF ULSAN FOUNDATION FOR INDUSTRY COOPERATION, Nam-gu Ulsan (KR)

(72) Inventors: Hyunsuk SUH, Seoul (KR); Joon-Pio HONG, Seoul (KR); Changsik PAK, Seoul (KR); Sanghoon LEE, Daegu (KR); Youngjoon CHO, Daegu (KR); Heejae SHIN, Daegu (KR)

(21) Appl. No.: 19/114,275

(22) PCT Filed: Dec. 28, 2022
(86) PCT No.: PCT/KR2022/021448

§ 371 Date: Feb. 4, 2026

(30) Foreign Application Priority Data
Sep. 22, 2022 (KR) 10-2022-0120072

(51) Int. Cl. A61N 1/05; A61N 1/36

(52) U.S. Cl. CPC A61N 1/0558; A61N 1/0556; A61N 1/36003

(57) ABSTRACT

A regenerative peripheral nerve interface device comprising: nerve electrodes for acquiring neural signals from peripheral nerves; muscle electrodes for acquiring muscle signals from muscle tissue; channel electrodes for electrically connecting the neural electrodes and the muscle electrodes to circuit terminals; and a shape-memory polymer covering at least a part of the neural electrodes, the muscle electrodes, and the channel electrodes.

### TECHNICAL FIELD

The present disclosure relates to a regenerative peripheral nerve interface device and a method for manufacturing the same. This study is related to “Development of bio-signal recognition bionic interface for intuitive control of upper and lower limb robots” (Project Unique Number: 1711174509, Project Number: KD000196) of the Pan-Ministry Full-cycle Medical Device Research and Development Project (R&D) supported by the Pan-Ministry Full-cycle Medical Device Research and Development Project Group funded by the Ministry of Science and ICT (government of Korea) in 2022.

### BACKGROUND ART

In general, amputation disabilities in the body can be congenital or acquired due to diseases such as arteriosclerosis, diabetes, and Buerger's disease, trauma from traffic accidents or industrial injuries, infections, tumors, and congenital malformations.

Most physical amputation disabilities can be rehabilitated through the use of prosthetic arms or legs, and assistive devices, along with proper training. However, rehabilitation for amputees has limitations in that, except for very simple tasks, they cannot feel the sensation of touching real objects or perform complex tasks such as writing. Artificial arms or legs used for amputees determine movement intentions through body power or surface electromyography, but they are often rejected by patients due to low recognition rates and malfunctions.

Accordingly, there is a demand for the development of technologies that enable users to move artificial arms and legs with their intension by utilizing the nerves remaining in the amputated area, and for this purpose, technologies for a regenerative peripheral nerve interface (RPNI) are being studied.

When applying a regenerative peripheral nerve interface (RPNI) to a patient with limb amputation, an intramuscular wire electrode is implanted to penetrate the muscle portion of the regenerative peripheral nerve interface. The regenerative peripheral nerve interface collects and analyzes signals from the muscle portion, which can be applied to neuroprosthetics, enabling intention-based control of the neuroprosthetics.

In conventional regenerative peripheral nerve interfaces, implanting a wire electrode that penetrates the muscle to obtain signals provides the advantage of improved signal-to-noise ratio, but this may cause damage to muscle tissue. In addition, in the operation of bionic arms or legs, not only signal acquisition but also sensory transmission through neural stimulation is required, but the conventional method has limitations in sensory transmission.

While intuitive operation of the bionic arm is possible through signals from the regenerative peripheral nerve interface, the signals from the regenerative peripheral nerve interface are somewhat different in form from actual muscle signals and actual neural signals. Therefore, a technology for sensory transmission through neural stimulation along with recording of neural signals with more information is required.

### PRIOR ART DOCUMENT

Patent Document 1: Korean Patent Application Publication No. 10-2013-0106639 (Published on Sep. 30, 2013)

### DETAILED DESCRIPTION OF INVENTION

### Technical Problems

Embodiments of the present disclosure have been conceived in view of the above background, and provide a regenerative peripheral nerve interface device and a method for manufacturing the same, which can simultaneously implement electrode contact with nerve and muscle tissue with a single interface through implantation of a peripheral nerve interface and muscle tissue.

### Technical Solution

In accordance with one embodiment of the present disclosure, a regenerative peripheral nerve interface device comprises: nerve electrodes for acquiring neural signals from a peripheral nerve; muscle electrodes for acquiring muscle signals from muscle tissue; channel electrodes for electrically connecting the nerve electrode and the muscle electrode to circuit terminals; and a shape memory polymer covering at least a portion of the nerve electrode, the muscle electrode, and the channel electrodes.

Further, the shape memory polymer may include: a binding-type body part in which the nerve electrodes are arranged and a binding hole is formed; a binding-type head part in which the muscle electrodes are arranged and whose side edges are sloped to allow insertion into the binding hole; and a binding-type connecting part connecting the binding-type body part and the binding-type head part.

Further, the binding hole may be formed to be smaller than a width of a base portion of the binding-type head part connected to the binding-type connecting part and larger than a width of a tip portion of the binding-type head part, and the binding-type head part may be configured to be insertable into and fixable in the binding hole when the binding-type body part wraps around the peripheral nerve.

Further, the shape memory polymer may include: a non-binding-type body part in which the nerve electrodes are arranged and which provides a plurality of body holes for allowing the peripheral nerve to pass through; and a non-binding-type head part in which the muscle electrodes are arranged, which is formed to extend from one end of the non-binding-type body part, and which provides a head hole arranged in a direction parallel to an arrangement direction of the plurality of body holes.

Further, the shape memory polymer may further include a non-binding-type tail part in which the circuit terminals are arranged, the non-binding-type tail part formed to extend from one side of the non-binding-type body part in a direction perpendicular thereto.

Further, in the non-binding-type body part, the nerve electrodes may be arranged as a pair between the plurality of body holes, and channel electrodes connected to different nerve electrodes may be arranged to be spaced apart from each other, with the plurality of body holes positioned therebetween.

Further, in the non-binding-type head part, the head hole may be formed to have a width at least smaller than a spacing between the paired nerve electrodes.

Further, the regenerative peripheral nerve interface device may further comprise a conductive material electrodeposited on the nerve electrode.

Further, the conductive material may include at least one of iridium oxide (IrO₂), gold (Au), carbon nanotubes, platinum black (Pt-black), titanium nitride, PEDOT: PSS conductive polymer, and molybdenum.

Further, the shape memory polymer may include: a first polymer layer coated on one surface of the nerve electrodes, the muscle electrodes, and the channel electrodes; and a second polymer layer coated on the other surface of the nerve electrodes, the muscle electrodes, and the channel electrodes.

Further, the second polymer layer may be etched to expose at least a portion of the nerve electrodes and the muscle electrodes.

Further, the nerve electrodes and the muscle electrodes may be configured as paired bipolar electrodes that are arranged in a crosswise direction in the shape memory polymer, and are positioned perpendicularly to each other when arranged to wrap around the peripheral nerve.

Further, a method for manufacturing a regenerative peripheral nerve interface device may comprise: forming a first polymer layer by initially coating a solution of a shape memory polymer on a wafer coated with a sacrificial layer; forming a metal electrode layer including nerve electrodes, muscle electrodes, and channel electrodes on the first polymer layer; forming a second polymer layer by secondarily coating the solution of shape memory polymers on the metal electrode layer; etching the second polymer layer to expose at least a portion of the nerve electrodes and the muscle electrodes; and etching the shape memory polymer into a desired shape.

Further, the method may further comprise electrodepositing a conductive material on the exposed nerve electrodes and muscle electrodes after the etching the second polymer layer.

Further, the etching the shape memory polymer into a desired shape may include etching the shape memory polymer into a buckle-shaped binding-type polymer, and the binding-type polymer may include a binding-type body part in which the nerve electrodes are arranged and a binding hole is formed, a binding-type head part in which the muscle electrodes are arranged and whose side edges are sloped to allow insertion into the binding hole, and a binding-type connecting part connecting the binding-type body part and the binding-type head part.

Further, the etching the shape memory polymer into a desired shape may include etching the shape memory polymer into a non-binding-type polymer, and the non-binding-type polymer may include a non-binding-type body part in which the nerve electrodes are arranged and which provides a plurality of body holes for allowing the peripheral nerve to pass through, and a non-binding-type head part in which the muscle electrodes are arranged, which is formed to extend from one end of the non-binding-type body part, and which provides a head hole arranged on a same extension line as the plurality of body holes.

Further, the conductive material may include at least one of iridium oxide (IrO₂), gold (Au), carbon nanotubes, platinum black (Pt-black), titanium nitride, PEDOT:PSS conductive polymer, and molybdenum.

### Effect of Invention

According to the embodiments of the present disclosure, by inserting muscle tissue into the severed nerve, it is possible to significantly reduce the formation of a neuroma in the severed nerve, and to obtain a biologically amplified signal from the muscle tissue.

Further, according to the embodiments of the present disclosure, neuroma-related pain can be reduced, and from a neuroengineering perspective, muscle tissue transplanted into the nerve can amplify neural signals, and the amplified neural signals can facilitate acquisition of neural signals and muscle signals, which enables better control of the neuroprosthetics utilizing the signals.

Furthermore, according to the embodiments of the present disclosure, the regenerative peripheral nerve interface device can be implanted into peripheral nerves and muscle tissues to enable simultaneous electrode contact with the peripheral nerves and muscle tissues, so that it is possible to perform neural stimulation and neural signal recording, and signal recording of the regenerative peripheral nerve interface (RPNI) and RPNI stimulation through the single interface device.

In addition, according to the embodiments of the present disclosure, by replacing the electrode portion for signal recording with the multi-channel nerve electrode using the shape memory polymer, it becomes possible to simultaneously record neural signals and RPNI signals, and to investigate the correlation between neural signals and RPNI signals.

Further, according to the embodiments of the present disclosure, from the perspective of intent-based neuroprosthetic operation, simultaneous recording of RPNI neural signals and muscle signals provides twice the input data compared to muscle signal recording alone, which enables more accurate and precise neuroprosthetic operation, and enables sensory transmission through neural stimulation through the channel of the nerve electrode in contact with the nerve, thereby enabling the control of the neuroprosthetics through sensory feedback in the intent-based neuroprosthetics.

### BRIEF DESCRIPTION OF THE DRAWINGS

FIG. 1 is a schematic diagram illustrating a regenerative peripheral nerve interface device (binding-type interface device) according to one embodiment of the present disclosure.

FIG. 2 is a cross-sectional view taken along line “II-II” of FIG. 1.

FIGS. 3 to 5 are state diagrams illustrating a manufacturing process of the regenerative peripheral nerve interface device (binding-type interface device) according to one embodiment of the present disclosure.

FIGS. 6 to 10 are state diagrams illustrating an implantation process of the regenerative peripheral nerve interface device according to one embodiment of the present disclosure into peripheral nerve and muscle tissue.

FIG. 11 is a schematic diagram illustrating a regenerative peripheral nerve interface device (non-binding-type interface device) according to another embodiment of the present disclosure.

FIG. 12 is a state diagram illustrating an implantation process of the regenerative peripheral nerve interface device according to another embodiment of the present disclosure into peripheral nerve and muscle tissue.

FIG. 13 is a schematic diagram illustrating a regenerative peripheral nerve interface device (non-binding-type interface device) according to a modification of another embodiment of the present disclosure.

FIG. 14 is a state diagram illustrating a process of implanting a regenerative peripheral nerve interface device into peripheral nerve and muscle tissue according to a modification of another embodiment of the present disclosure.

FIG. 15 is a block diagram illustrating a method of manufacturing a regenerative peripheral nerve interface device according to the present disclosure.

### BEST MODE FOR CARRYING OUT THE INVENTION

Hereinafter, specific embodiments for implementing the technical idea of the present disclosure will be described in detail with reference to the drawings.

In addition, in describing the present disclosure, when it is determined that detailed descriptions of known configurations or functions may obscure the gist of the present disclosure, the detailed descriptions will be omitted.

Moreover, it should be understood that when a component is referred to as being ‘connected to’, ‘supported by’, ‘accessed to’, ‘provided to’, ‘transmitted to’ or ‘contacted with’ another component, it may be directly connected to, supported by, accessed to, provided to, transmitted to or contacted with another component, but other components may exist between the components.

The terms used in the present specification are only used for describing the specific embodiments and are not intended to limit the present disclosure. Singular expressions include plural expressions unless the context clearly indicates otherwise.

In addition, in the present specification, expressions such as upper, lower, side, etc. are described based on the drawings, and it is made clear in advance that they may be expressed differently if the direction of the object is changed. For the same reason, some components in the accompanying drawings are exaggerated, omitted, or schematically illustrated, and the size of each component does not entirely reflect the actual size.

Further, terms including ordinal numbers, such as first, second, etc., may be used to describe various components, but the components are not limited by these terms. These terms are only used to distinguish one component from another.

The meaning of “including” used in the present specification specifies specific features, regions, integers, steps, operations, elements and/or components, and does not exclude the presence or addition of other specific features, regions, integers, steps, operations, elements, components, and/or groups.

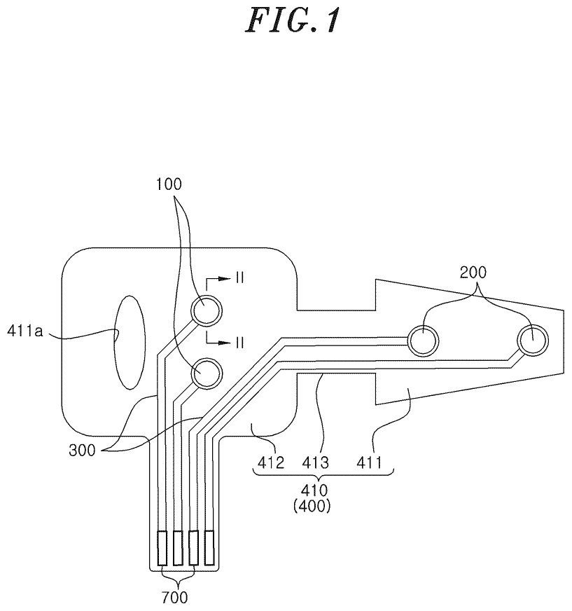

Hereinafter, a specific configuration of a regenerative peripheral nerve interface device according to the present disclosure will be described with reference to FIGS. 1 to 14.

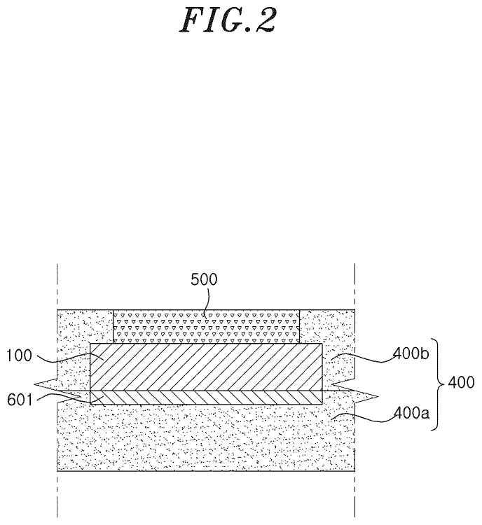

Referring to FIGS. 1 and 2, a regenerative peripheral nerve interface device 10 according to one embodiment of the present disclosure is capable of performing neural stimulation and neural signal recording through electrode contact with nerves through implantation into a peripheral nerve 31 and muscle tissue, and can provide shape memory effects and thermal softening effects depending on temperature changes according to the purpose by utilizing a shape memory polymer 400. The regenerative peripheral nerve interface device 10 may include a nerve electrode 100, a muscle electrode 200, a channel electrode 300, and a shape memory polymer 400.

Specifically, the nerve electrode 100 can acquire neural signals from the peripheral nerve 31. The nerve electrode 100 may be paired bipolar electrodes. The nerve electrode 100 may be arranged in a crosswise direction relative to the muscle electrode 200 in the shape memory polymer 400. For example, an imaginary extension line of the paired nerve electrodes 100 may intersect with an imaginary extension line of the paired muscle electrodes 200.

To perform bipolar recording of neural signals, the distance between the paired nerve electrodes 100 may be set to 2 mm or more. In addition, for stable neural signal recording, the impedance of the nerve electrode 100 may be within 10 kΩ at a target neural signal frequency of 1 kHz. More preferably, the impedance of the nerve electrode 100 may be as low as about 1 to 2 kΩ. When the impedance of the nerve electrode 100 is higher than 10 kΩ, a background noise signal may be recorded at a high level, resulting in a lower signal-to-noise-ratio (SNR). A higher signal-to-noise ratio (SNR) indicates a higher-quality recording performance. In the present embodiment, it is preferred that the impedance of the nerve electrode 100 be 10 kΩ or less at the target neural signal frequency of 1 kHz, but recording of the neural signal is possible even when the impedance of the nerve electrode 100 is within 100 kΩ.

If the size of the nerve electrode 100 is smaller than a predetermined size, the neural signal at a localized site in contact with the nerve can be recorded, so that recording selectivity can be improved. However, as the size of the nerve electrode 100 is getting smaller than the predetermined size, the impedance value of the nerve electrode 100 may increase. Therefore, it is preferable to select the size and impedance of the nerve electrode 100 considering the material of the nerve electrode 100. Although the nerve electrode 100 is generally planer, the nerve electrode 100 may be formed in various shapes including three-dimensional structures, depending on the required contact mode with nerve.

The nerve electrode 100 may be formed of gold (Au), which is a biocompatible material. As a matter of course, the nerve electrode 100 may be formed of materials other than gold (Au), and additional coating may be applied to enhance conductivity. In addition, the nerve electrode 100 may be deposited on the shape memory polymer 400 by increasing its thickness, thereby improving contact.

The nerve electrode 100 may have a diameter of 1 mm to secure sufficiently low impedance. Considering the additional coating process and the like, it is preferable for the nerve electrode 100 to have a size of at least 50 μm or more.

The nerve electrode 100 may be additionally coated with a conductive material 500. When an additional coating of the conductive material 500 is applied to the nerve electrode 100, the impedance value can be sufficiently lowered even in a small-sized nerve electrode 100. For example, when the conductive material 500 such as iridium oxide (IrO₂) is additionally coated on the nerve electrode 100, the impedance of the nerve electrode 100 can be significantly lowered through this additional coating. As a result of the iridium oxide coating, it has been confirmed that the impedance of the nerve electrode 100 was reduced by a factor of 5, and the charge storage capacity was increased by about a factor of 11, indicating a significant performance improvement.

In the present embodiment, iridium oxide (IrO2) is used as the conductive material 500, but besides iridium oxide (IrO2), various types of conductive materials 500 capable of lowering impedance may be used. For example, as the conductive material 500, gold (Au), carbon nanotubes, platinum black (Pt-black), titanium nitride, PEDOT: PSS (polystyrene sulfonic acid doped poly(3,4-ethylenedioxythiophene)) conductive polymer, molybdenum, etc. may be used.

The muscle electrode 200 can acquire muscle signals from muscle tissue. The muscle electrode 200 may be paired bipolar electrodes. To perform bipolar recording of muscle signals, the paired muscle electrodes 200 can stably acquire muscle signals when the distance between the paired muscle electrodes 200 is at least 2 mm. As a matter of course, the distance between the muscle electrodes 200 may vary depending on the size and orientation of the muscle tissue.

The muscle electrode 200 may be arranged in a crosswise direction relative to the nerve electrode 100 in the shape memory polymer 400. For example, the imaginary extension line of the paired muscle electrodes 200 may intersect with the imaginary extension line of the paired nerve electrodes 100. Since the nerve electrodes 100 and the muscle electrodes 200 are arranged to intersect in the shape memory polymer 400, when the regenerative peripheral nerve interface device 10 wraps around the peripheral nerve 31, the nerve electrodes 100 and the muscle electrodes 200 may be positioned perpendicularly to each other with respect to the peripheral nerve 31. When the nerve electrodes 100 and the muscle electrodes 200 are positioned perpendicularly to each other with respect to the peripheral nerve 31, the recording of the signal and the selectivity of the stimulation can be improved.

Although the nerve electrode 100 and the muscle electrode 200 are described as bipolar electrodes in the present embodiment, the nerve electrode 100 and the muscle electrode 200 may also be configured as various other types of electrodes capable of effectively acquiring neural signals and muscle signals. For example, the nerve electrode 100 and the muscle electrode 200 may be monopolar electrodes, tripolar electrodes, or multipolar electrodes. In the nerve electrode 100 other than the bipolar electrode, the distance between the paired nerve electrodes 100 is preferably at least 2 mm, and more preferably, the distance between the paired nerve electrodes 100 may be an optimized distance of about 4 mm.

The channel electrode 300 can electrically connect the nerve electrode 100 and the muscle electrode 200 to a circuit terminal 700. The circuit terminal 700 may be included in a flexible printed circuit board (FPCB) which is made of a flexible insulating substrate. The circuit terminal 700 can record neural signals and muscle signals. The flexible printed circuit board may be bonded with anisotropic conductive film (ACF) for electrical connection of very thin films.

The channel electrode 300 can transmit the neural signals and muscle signals acquired from the nerve electrode 100 and the muscle electrode 200 to the circuit terminal 700. The channel electrode 300 may be arranged in various shapes in the shape memory polymer 400 according to the desired shape of the shape memory polymer 400. By setting the thickness of the channel electrode 300 to be sufficiently wide, e.g., 0.25 mm or more, conductivity and stability can be secured.

The shape memory polymer 400 can serve not only to define the overall shape of the electrode, but also to insulate the conductive electrodes such as the nerve electrode 100 and the muscle electrode 200. In addition, the shape memory polymer 400 can be opened (patterned) at desired regions to enable contact and connection with the nerve electrode 100 or the muscle electrode 200.

The shape memory polymer 400 may be configured as a polymer layer that encloses at least a portion of the nerve electrode 100, the muscle electrode 200, and the channel electrode 300. For example, the shape memory polymer 400 may include a first polymer layer 400a coated on one surfaces of the nerve electrode 100, the muscle electrode 200, and the channel electrode 300, and a second polymer layer 400b coated on the other surfaces of the nerve electrode 100, the muscle electrode 200, and the channel electrode 300.

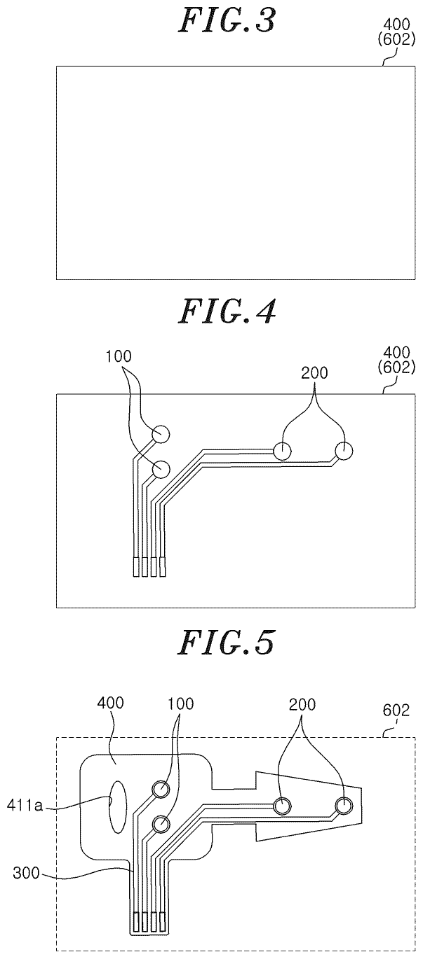

Referring to FIG. 3, the first polymer layer 400a can be formed by spin-coating a solution of the shape memory polymer 400 on a wafer coated with a sacrificial layer 602 of aluminum thin film (1 μm). The solution of the shape memory polymer 400 may be prepared by mixing the monomers of 1,3,5-Triallyl-1,3,5-triazine-2,4,6(1H,3H,5H)-trione (TATATO), trimethylolpropane tris(3-mercaptopropionate) (TMTMP), and tricyclodecane dimethanol diacrylate (TCMDA), and 2-dimethoxy-2-phenyl-acetophenone (DMPA) which is a photoinitiator for photopolymerization, at an appropriate ratio. The solution of the shape memory polymer 400 can be polymerized through photopolymerization by irradiating ultraviolet rays having a wavelength of 365 nm for 30 minutes.

Referring to FIG. 4, once the first polymer layer 400a is formed, an adhesion layer 601 (chromium (Cr) or titanium (Ti)) and a metal conductive layer (the nerve electrode 100, the muscle electrode 200, the channel electrode 300, and the like) can be micro-patterned on the first polymer layer 400a through a standard photolithography process using photoresist and sputtering. Once the adhesion layer and the metal conductive layer are micro-patterned, the second polymer layer 400b can be formed on the metal conductive layer and the first polymer layer 400a by spin-coating the solution of the shape memory polymer 400.

Referring to FIG. 5, once the second polymer layer 400b is formed, the shape memory polymer 400 can be etched and micropatterned into a desired shape by performing patterning using a hard mask and reactive ion etching using oxygen plasma. Then, when the sacrificial layer (Al) is dissolved by the etchant, the regenerative peripheral nerve interface device can be obtained. In the present embodiment, the shape memory polymer 400 can be etched into a binding-type polymer 410 or a non-binding-type polymer 420.

The shape memory polymer 400 may be provided as a binding-type polymer 410 that can wrap around the peripheral nerve 31 in a buckle shape. The binding-type polymer 410 may include a binding-type body part 411, a binding-type head part 412, and a binding-type connecting part 413.

The binding-type body part 411 may include the first polymer layer 400a and the second polymer layer 400b, on which the nerve electrode 100 and the channel electrode 300 can be arranged. A binding hole 411a may be formed in the binding-type body part 411. The circuit terminal 700 may be arranged in the binding-type polymer 410. The binding-type body part 411 may be integrally connected to the binding-type head part 412 through the binding-type connecting part 413. The binding hole 411a may be formed in the binding-type body part 411.

When the binding-type polymer 410 wraps around the peripheral nerve 31, the size (diameter) of the binding hole 411a may be selected to stably press and fix the nerve toward the electrodes in consideration of the distance between the electrodes, the sizes of the electrodes, and the like. For example, to achieve stable buckling fixation between the binding-type head part 412 and the binding hole 411a of the binding-type body part 411, the binding hole 411a may be formed to be smaller than the width of a base portion of the binding-type head part 412 connected to the binding-type connecting part 413 and larger than the width of a tip portion of the binding-type head part 412. For example, when the width of the base portion of the binding-type head part 412 is 4 mm and the width of the tip portion of the binding-type head part 412 is 2 mm, the size (diameter) of the binding hole 411a may be 3 mm.

The binding-type head part 412 may include the first polymer layer 400a and the second polymer layer 400b, on which the muscle electrode 200 and the channel electrode 300 are arranged. The binding-type head part 412 may have side edges that are sloped to allow insertion into the binding hole 411a. The side edges of the binding-type head part 412 may be sloped so that a width between the side edges gradually narrows in a direction away from the binding-type body part 411. When the binding-type polymer 410 wraps around the peripheral nerve 31, the binding-type head part 412 can be inserted into the binding hole 411a of the binding-type body part 411 and fixed through buckling.

The binding-type connecting part 413 may include the first polymer layer 400a and the second polymer layer 400b, on which the channel electrode 300 can be arranged. The binding-type connecting part 413 may integrally connect the binding-type body part 411 and the binding-type head part 412. The width of one end of the binding-type connecting part 413 connected to the binding-type body part 411 may be the same as the width of one end of the binding-type connecting part 413 connected to the binding-type head part 412.

Hereinafter, a process for implanting the regenerative peripheral nerve interface device having the configuration described above into a peripheral nerve and muscle tissue will be described.

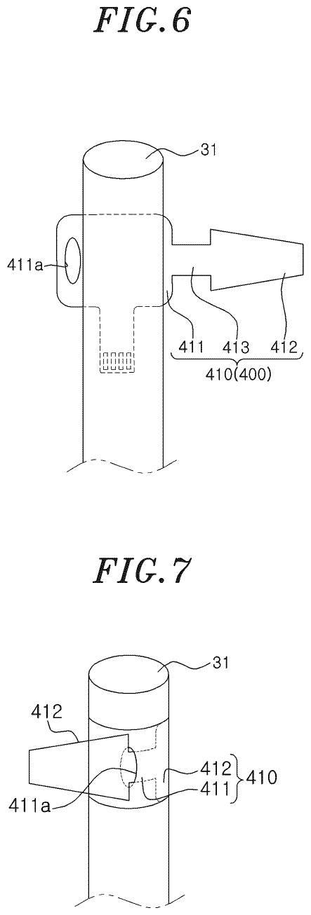

Referring to FIG. 6, in order to implant the regenerative peripheral nerve interface device 10 into the peripheral nerve 31 and muscle tissue 32, the shape memory polymer 400 is first placed so that the muscle electrode 200 and the second polymer layer 400b face upward, and then the peripheral nerve 31 is then positioned on the upper surface of the shape memory polymer 400.

Referring to FIG. 7, once the peripheral nerve 31 is positioned on the upper surface of the shape memory polymer 400, the shape memory polymer 400 is wrapped around the outer surface of the peripheral nerve 31 in one direction (to the left in the drawing), while the binding-type head part 412 of the shape memory polymer 400 is inserted through the binding hole 411a of the binding-type body part 411. The binding-type head part 412 inserted through the binding hole 411a of the binding-type body part 411 can be caught and restrained by the binding hole 411a. At this time, the lower surface of the shape memory polymer 400 is positioned facing upward.

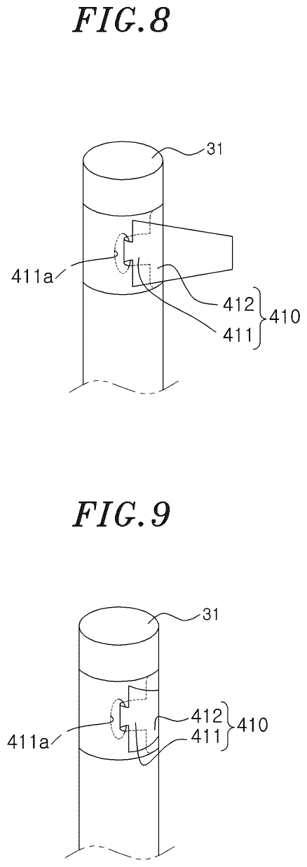

Referring to FIG. 8, once the binding-type head part 412 is inserted through the binding hole 411a of the binding-type body part 411, the binding-type head part 412 is bent 180 degrees in the other direction (to the right in the drawing).

Referring to FIG. 9, in the state where the binding-type head part 412 is bent 180 degrees in the other direction (to right side in the drawing), and the binding-type head part 412 is wound tightly against the peripheral nerve 31. At this time, since the upper surface of the shape memory polymer 400 is positioned facing upward, the muscle electrode 200 remains in a state where it can make contact with the muscle tissue 32.

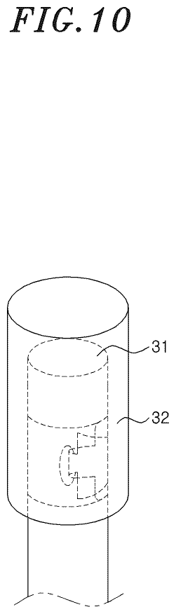

Referring to FIG. 10, in the state where the binding-type head part 412 is wound tightly against the peripheral nerve 31, the muscle tissue 32 is wrapped over the end of the peripheral nerve 31, thereby completing the implantation of the regenerative peripheral nerve interface device 10 to the peripheral nerve 31 and the muscle tissue 32.

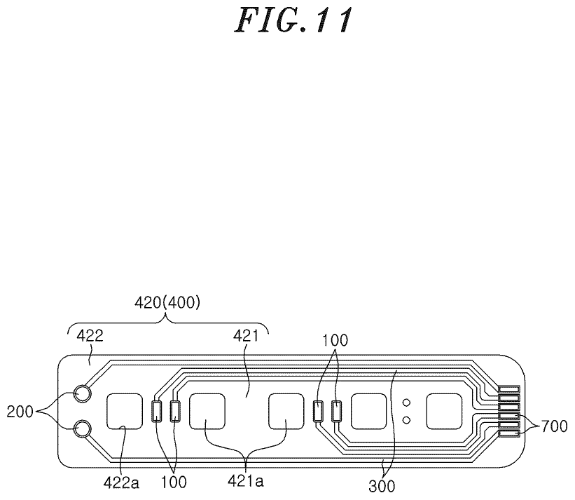

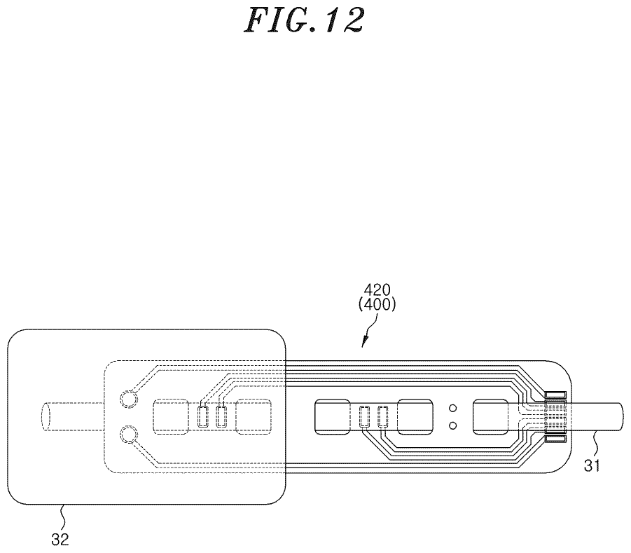

Meanwhile, referring to FIGS. 11 and 12, in another embodiment of the present disclosure, the shape memory polymer 400 may be provided as a non-binding-type polymer 420 having an overall straight shape. The non-binding-type polymer 420 may include a non-binding-type body part 421 and a non-binding-type head part 422.

The non-binding-type body part 421 may include a first polymer layer 400a and a second polymer layer 400b, on which the nerve electrode 100, the channel electrode 300, and the circuit terminal 700 can be arranged. The paired nerve electrodes 100 may be arranged to be spaced apart by a distance of 1 mm or more. In the present embodiment, the distance between the paired nerve electrodes 100 may be 2 mm. In addition, the paired nerve electrodes 100 may be provided as multiple paired nerve electrodes 100 spaced apart from each other by a predetermined distance with a body hole 421a positioned on the same line therebetween. The distance between the pairs of nerve electrodes 100 spaced apart from each other with the body hole 421a therebetween may be 8.15 mm.

A plurality of body holes 421a through which peripheral nerves 31 can pass may be formed in the non-binding-type body part 421. The plurality of body holes 421a may be positioned on the same line in the non-binding-type body part 421. The body hole 421a may have a diameter at least larger than the diameter of the peripheral nerve 31 to allow insertion of the peripheral nerve 31. The non-binding-type head part 422 may be integrally formed to extend from one end of the non-binding-type body part 421. In the non-binding-type body part 421, channel electrodes 300 connected to different nerve electrodes 100 may be arranged above and below, spaced apart with the plurality of body holes 421a therebetween.

In order for the peripheral nerve 31 to stably pass through and be fixed in the body hole 421a, the non-bound body part 421 may have body holes 421a formed with the nerve electrode 100 therebetween. An additional body hole 421a may be formed in the non-bound body part 421. The size of the body hole 421a may be the minimum size necessary to allow the peripheral nerve 31 to pass through. In addition, in order to stably press and fix the peripheral nerve inserted into the body hole 421a in the direction where the electrode is located, the distance between the body hole 421a and the electrode and the size of the electrode may be adjusted.

After the peripheral nerve 31 penetrates the non-bound polymer 420, when the non-bound polymer 420 is wrapped by muscle tissue, at least some of the nerve electrodes 100 among the plurality of nerve electrodes 100 can be wrapped by the muscle tissue, and the remaining nerve electrodes 100 can be exposed to the outside. In this case, since the nerve electrodes 100 are located inside and outside the muscle tissue, the difference in neural signal recording and stimulation between the inside and outside of the muscle tissue can be verified.

The non-binding-type head part 422 may include the first polymer layer 400a and the second polymer layer 400b, on which the muscle electrodes 200 and the channel electrodes 300 are arranged. The paired muscle electrodes 200 may be arranged to be spaced apart by a distance of 1 mm or more. In the present embodiment, the distance between the paired muscle electrodes 200 may be 1 mm. Although it may vary depending on the size and orientation of muscle tissue, the muscle electrodes 200 can stably acquire muscle signals using a differential bipolar recording method at a distance of at least 2 mm or more.

A head hole 422a through which the peripheral nerve 31 can pass may be formed in the non-binding-type head part 422. The head hole 422a may be positioned on the same line as the plurality of body holes 421a. The shape of the head hole 422a may correspond to the shape of the body hole 421a. The head hole 422a may have a width at least smaller than the spacing between the paired nerve electrodes 100.

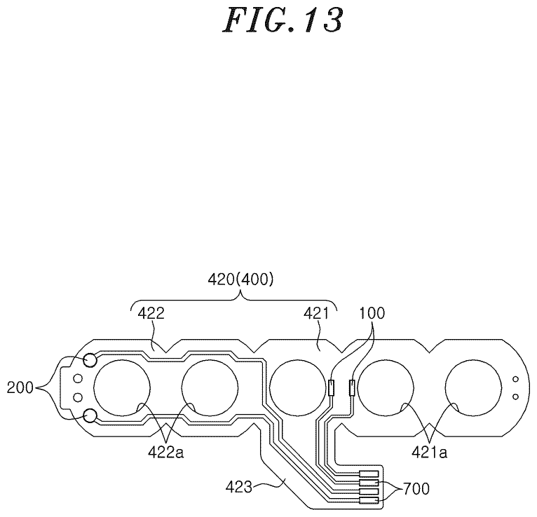

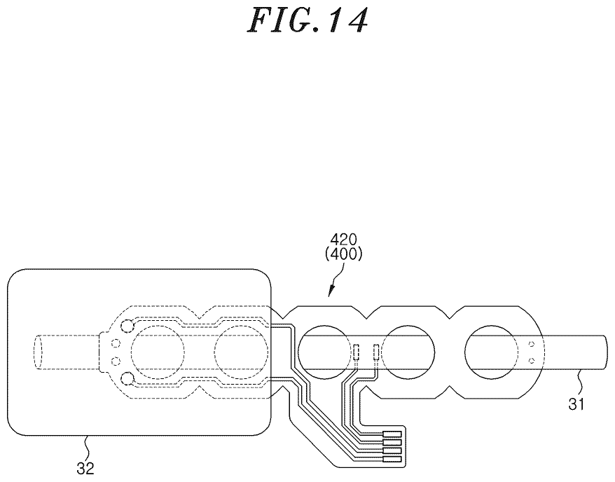

Referring to FIGS. 13 and 14, according to a modified example of another embodiment of the present disclosure, the shape memory polymer 400 may be provided as a non-binding-type polymer 420 having a figure-eight shape as a whole. The shape memory polymer 400 may include a non-binding-type body part 421, a non-binding-type head part 422, and a non-binding-type tail part 423.

The non-binding-type body part 421 may include the first polymer layer 400a and the second polymer layer 400b, on which the nerve electrode 100 and the channel electrode 300 can be arranged. The paired nerve electrodes 100 may be spaced apart by a distance of 1 mm or more. In the present embodiment, the distance between the paired nerve electrodes 100 may be 1.2 mm.

The non-binding-type body part 421 may be formed with a plurality of body holes 421a through which the peripheral nerve 31 can pass. The plurality of body holes 421a may be positioned on the same line in the non-binding-type body part 421. The non-binding-type head part 422 may be formed integrally to extend from one end of the non-binding-type body part 421. The non-binding-type tail part 423 may be formed integrally to extend from a side of the non-binding-type body part 421.

The non-binding-type head part 422 may include the first polymer layer 400a and the second polymer layer 400b, on which the muscle electrodes 200 and the channel electrodes 300 are arranged. A head hole 422a through which the peripheral nerve 31 can pass may be formed in the non-binding-type head part 422. The head hole 422a may be positioned on the same line as the plurality of body holes 421a. The shape of the head hole 422a may correspond to the shape of the body hole 421a.

The non-binding-type tail part 423 may include the first polymer layer 400a and the second polymer layer 400b, on which the circuit terminal 700 and the channel electrode 300 can be arranged. The non-binding-type tail part 423 may be formed integrally to extend from one side of the non-binding-type body part 421 in a direction perpendicular thereto.

In the case of the straight-shaped non-binding-type polymer 420, the circuit terminal 700 is arranged on the same extension line as the body hole 421a, whereas in the case of the figure-eight-shaped non-binding-type polymer 420, the circuit terminal 700 is arranged on the non-binding-type tail part 423 located in a direction perpendicular to the extension line of the body hole 421a. In the case of the figure-eight-shaped non-binding-type polymer 420, since the non-binding-type tail part 423 protrudes from the side of the non-binding-type body part 421, there is a possibility of damage due to non-binding. However, since the circuit terminal 700, which is relatively thickly packaged, is located on the non-binding-type tail part 423, direct contact between the circuit terminal 700 and the peripheral nerve 31 can be prevented.

Hereinafter, a method for manufacturing the regenerative peripheral nerve interface device having the configuration described above will be described.

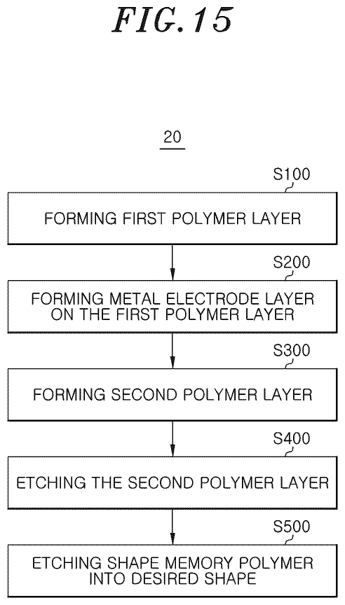

Referring to FIG. 15, a method 20 for manufacturing the regenerative peripheral nerve interface device according to the present disclosure may include a step of forming a first polymer layer (S100), a step of forming a metal electrode layer on the first polymer layer (S200), a step of forming a second polymer layer (S300), a step of etching the second polymer layer (S400), and a step of etching a shape memory polymer into a desired shape (S500).

In the step S100 of forming the first polymer layer, the first polymer layer may be formed by primarily coating a solution of shape memory polymer on a wafer coated with a sacrificial layer. The solution of shape memory polymer may be prepared by mixing three types of monomers, 1,3,5-Triallyl-1,3,5-triazine-2,4,6(1H,3H,5H)-trione (TATATO), trimethylolpropane tris(3-mercaptopropionate) (TMTMP), and tricyclodecane dimethanol diacrylate (TCMDA), and 2-dimethoxy-2-phenyl-acetophenone (DMPA) which is a photoinitiator for photopolymerization, at an appropriate ratio.

In the step S200 of forming the metal electrode layer on the first polymer layer, a metal electrode layer including a nerve electrode, a muscle electrode, and a channel electrode may be formed on the first polymer layer.

For example, once the first polymer layer is formed on the wafer coated with the sacrificial layer, the metal electrode layer may be formed on the first polymer layer by micro-patterning an adhesive layer and a metal conductive layer on the first polymer layer through a standard photolithography process using a photoresist and sputtering.

In the step S300 of forming the second polymer layer, the second polymer layer may be formed by secondarily coating the solution of shape memory polymer on the metal electrode layer. Once the metal electrode layer is formed on the first polymer layer, the solution of shape memory polymer may be spin-coated on the metal conductive layer and the first polymer layer, thereby forming the second polymer layer on the metal conductive layer and the first polymer layer.

In the step S400 of etching the second polymer layer, the second polymer layer may be partially etched. Through etching of the second polymer layer, at least a portion of the nerve electrode and the muscle electrode may be exposed.

After the step S400 of etching the second polymer layer S400, a conductive material may be electrodeposited on the exposed nerve electrode and muscle electrode. When the nerve electrode and muscle electrode are additionally coated with the conductive material, the impedance value can be sufficiently reduced even in a small-sized electrode. In this case, the conductive material that can be used may include iridium oxide (IrO₂), gold (Au), carbon nanotubes, platinum black (Pt-black), titanium nitride, PEDOT: PSS conductive polymer, molybdenum, etc.

In the step S500 of etching the shape memory polymer into a desired shape, the shape memory polymer may be etched into a desired shape. In the present embodiment, the shape memory polymer may be etched into a binding-type polymer or a non-binding-type polymer. Thereafter, the sacrificial layer is dissolved through an etchant, completing the manufacturing of the regenerative peripheral nerve interface device.

After the manufacture of the regenerative peripheral nerve interface device is completed, the regenerative peripheral nerve interface device may be subjected to electrode packaging of the metal electrode layer. For electrode packaging of the regenerative peripheral nerve interface device, the metal electrode layer can be electrically connected to the circuit terminal of a flexible printed circuit board using anisotropic conductive film bonding (ACF bonding). Then, after connecting the flexible printed circuit board and the head port through biocompatible wires, the exposed portions of the biocompatible wires may be completely encapsulated through UV epoxy to block charge leakage.

As described above, the present disclosure has the following excellent advantages: the regenerative peripheral nerve interface device can be implanted into peripheral nerves and muscle tissues; simultaneous electrode contact with peripheral nerves is possible; it is possible to simultaneously record neural signals and RPNI signals and to investigate the correlation between neural signals and RPNI signals by replacing the electrode portion for signal recording with the multi-channel nerve electrode using the shape memory polymer; and from the perspective of intent-based neuroprosthetic operation, simultaneous recording of RPNI neural signals and muscle signals provides twice the input data compared to muscle signal recording alone, allowing more accurate and precise neuroprosthetic operation.

Although the embodiments of the present disclosure have been described as specific embodiments, this is merely an example, and the present disclosure should be construed as having the broadest scope according to the technical idea disclosed herein without being limited thereto. Those skilled in the art may implement a pattern of a shape not indicated herein by combining/substituting the disclosed embodiments, but this also does not deviate from the scope of the present disclosure. In addition, those skilled in the art may easily change or modify the disclosed embodiments based on the present specification, and it is clear that such changes or modifications also fall within the scope of the present disclosure.

1. A regenerative peripheral nerve interface device comprising:
nerve electrodes for acquiring neural signals from a peripheral nerve;
muscle electrodes for acquiring muscle signals from muscle tissue;
channel electrodes for electrically connecting the nerve electrode and the muscle electrode to circuit terminals; and
a shape memory polymer covering at least a portion of the nerve electrode, the muscle electrode, and the channel electrodes.

2. The regenerative peripheral nerve interface device of claim 1, wherein the shape memory polymer includes:
a binding-type body part in which the nerve electrodes are arranged and a binding hole is formed;
a binding-type head part in which the muscle electrodes are arranged and whose side edges are sloped to allow insertion into the binding hole; and
a binding-type connecting part connecting the binding-type body part and the binding-type head part.

3. The regenerative peripheral nerve interface device of claim 2, wherein the binding hole is formed to be smaller than a width of a base portion of the binding-type head part connected to the binding-type connecting part and larger than a width of a tip portion of the binding-type head part, and
the binding-type head part is configured to be insertable into and fixable in the binding hole when the binding-type body part wraps around the peripheral nerve.

4. The regenerative peripheral nerve interface device of claim 1, wherein the shape memory polymer includes:
a non-binding-type body part in which the nerve electrodes are arranged and which provides a plurality of body holes for allowing the peripheral nerve to pass through; and
a non-binding-type head part in which the muscle electrodes are arranged, which is formed to extend from one end of the non-binding-type body part, and which provides a head hole arranged in a direction parallel to an arrangement direction of the plurality of body holes.

5. The regenerative peripheral nerve interface device of claim 4, wherein the shape memory polymer further includes a non-binding-type tail part in which the circuit terminals are arranged, the non-binding-type tail part formed to extend from one side of the non-binding-type body part in a direction perpendicular thereto.

6. The regenerative peripheral nerve interface device of claim 4, wherein in the non-binding-type body part, the nerve electrodes are arranged as a pair between the plurality of body holes, and
channel electrodes connected to different nerve electrodes are arranged to be spaced apart from each other, with the plurality of body holes positioned therebetween.

7. The regenerative peripheral nerve interface device of claim 4, wherein in the non-binding-type head part, the head hole is formed to have a width at least smaller than a spacing between the paired nerve electrodes.

8. The regenerative peripheral nerve interface device of claim 1, further comprising a conductive material electrodeposited on the nerve electrode.

9. The regenerative peripheral nerve interface device of claim 8, wherein the conductive material includes at least one of iridium oxide (IrO₂), gold (Au), carbon nanotubes, platinum black (Pt-black), titanium nitride, PEDOT: PSS conductive polymer, and molybdenum.

10. The regenerative peripheral nerve interface device of claim 1, wherein the shape memory polymer includes:
a first polymer layer coated on one surface of the nerve electrodes, the muscle electrodes, and the channel electrodes; and
a second polymer layer coated on the other surface of the nerve electrodes, the muscle electrodes, and the channel electrodes.

11. The regenerative peripheral nerve interface device of claim 10, wherein the second polymer layer is etched to expose at least a portion of the nerve electrodes and the muscle electrodes.

12. The regenerative peripheral nerve interface device of claim 1, wherein the nerve electrodes and the muscle electrodes are configured as paired bipolar electrodes that are arranged in a crosswise direction in the shape memory polymer, and are positioned perpendicularly to each other when arranged to wrap around the peripheral nerve.

13. A method for manufacturing a regenerative peripheral nerve interface device, the method comprising:
forming a first polymer layer by initially coating a solution of a shape memory polymer on a wafer coated with a sacrificial layer;
forming a metal electrode layer including nerve electrodes, muscle electrodes, and channel electrodes on the first polymer layer;
forming a second polymer layer by secondarily coating the solution of shape memory polymers on the metal electrode layer;
etching the second polymer layer to expose at least a portion of the nerve electrodes and the muscle electrodes; and
etching the shape memory polymer into a desired shape.

14. The method of claim 13, further comprising electrodepositing a conductive material on the exposed nerve electrodes and muscle electrodes after the etching the second polymer layer.

15. The method of claim 13, wherein the etching the shape memory polymer into a desired shape includes etching the shape memory polymer into a buckle-shaped binding-type polymer, and
the binding-type polymer includes a binding-type body part in which the nerve electrodes are arranged and a binding hole is formed, a binding-type head part in which the muscle electrodes are arranged and whose side edges are sloped to allow insertion into the binding hole, and a binding-type connecting part connecting the binding-type body part and the binding-type head part.

16. The method of claim 13, wherein the etching the shape memory polymer into a desired shape includes etching the shape memory polymer into a non-binding-type polymer, and
the non-binding-type polymer includes a non-binding-type body part in which the nerve electrodes are arranged and which provides a plurality of body holes for allowing the peripheral nerve to pass through, and a non-binding-type head part in which the muscle electrodes are arranged, which is formed to extend from one end of the non-binding-type body part, and which provides a head hole arranged on a same extension line as the plurality of body holes.

17. The method of claim 14, wherein the conductive material includes at least one of iridium oxide (IrO₂), gold (Au), carbon nanotubes, platinum black (Pt-black), titanium nitride, PEDOT:PSS conductive polymer, and molybdenum.
